(19) United States
(12) Patent Application Publication
(10) Pub. No.: US 2005/0227261 A1
(43) Pub. Date: Oct. 13, 2005

(54) Method for sequencing-by-synthesis

(76) Inventors: Nigel Tooke, Knivsta (SE); Peter Hagerlid, Uppsala (SE); Bjorn Ekstrom, Uppsala (SE)

Correspondence Address: MINTZ LEVIN COHN FERRIS GLOVSKY & POPEO, 666 THIRD AVENUE, NEW YORK, NY, 10017, US

(21) Appl. No.: 11/018,722

(22) Filed: Dec. 20, 2004

Related U.S. Application Data
(60) Provisional application No. 60/531,620, filed on Dec. 23, 2003.

(30) Foreign Application Priority Data
Dec. 22, 2003 (SE) SE0303473-3

(51) Int. Cl. C12Q 1/68; C12M 1/34

(52) U.S. Cl. 435/6; 435/287.2

(57) ABSTRACT

The invention refers to a tubular member, such as a pipette tip, for binding nucleic acid molecules for a subsequent biomolecular reaction, to methods for at least partly performing a sequencing-by-synthesis reaction in a tubular member, to an automated system using the tubular member for performing the methods of the invention, and kits and computer programs for use in relation to the methods of the invention. By performing a sequencing-by-synthesis reaction in a tubular member, such as a pipette tip, a new format for enzymatic reactions of this type, i.e. sequencing by synthesis, is provided.

### RELATED APPLICATIONS

This application claims the benefit of priority from U.S. application Ser. No. 60/531,620 filed Dec. 23, 2003 and from Swedish Application SE0303473-3 filed December 22, 2003; both applications are incorporated by reference herein in their entirety.

### TECHNICAL FIELD

The invention refers to a tubular member, such as a pipette tip, for binding nucleic acid molecules for a subsequent biomolecular reaction, to methods for at least partly performing a sequencing-by-synthesis reaction in a tubular member, as well as to an automated system using the tubular member for performing the methods of the invention.

### BACKGROUND

Sequencing-by-synthesis is a method for determining the identity of one or more nucleotides in a nucleic acid sample. An oligonucleotide primer is designed to anneal to a predetermined position of the sample template molecule. The primer/template is presented with a nucleotide in the presence of a polymerase enzyme. If the nucleotide is complementary to the position on the sample template molecule that is directly 3′ of the end of the oligonucleotide primer then the DNA polymerase will extend the primer with the nucleotide. The incorporation of the nucleotide and the identity of the inserted nucleotide can then be detected by e.g. the Pyrosequencing™ method. As a result of an enzymatic system this method will give rise to a detectable light signal that reflects the occurrence, identity and number of incorporations. Unincorporated nucleotides can then be removed and the next position in the primer/template can be queried with another nucleotide species.

Normally, the Pyrosequencing™ method is performed in an automated fashion by adding the sample and the necessary reagents to a well in a microtiter plate and performing the reactions in a homogeneous fashion.

However, by running a homogeneous reaction in a microtiter plate some problems will arise. The method is limited primarily by the accumulation of the products of out-of-phase primer extension, so-called ‘shift’. Shift is the result of incomplete or excessive extension of the primer due to, for example, the presence of sub-optimal levels of nucleotides. The homogeneous Pyrosequencing reaction involves a competition between the DNA polymerase that incorporates the correct nucleotide, and apyrase that degrades unincorporated nucleotides. If the level of correct nucleotide drops below the optimum for the DNA polymerase during the incorporation step then further extension of some primers will be incomplete. The result is a population of extended primer molecules that are one or more bases shorter than the correct, fully extended primers (so-called minus shift). This is particularly evident when stretches of several nucleotides of the same species (so-called homopolymer stretches) are to be incorporated. In this case incomplete extension is a prime limitation to the read-length. On the other hand, if apyrase is not sufficiently effective in degrading nucleotides, then excess, unincorporated nucleotides will remain in the reaction and be incorporated in certain situations when the next, correct nucleotide is presented and incorporated, and the sequence reaction permits further extension using the undesired nucleotide background (so-called plus shift). The success of the Pyrosequencing reaction therefore relies heavily on a fine balance between the concentration of apyrase, the concentration of nucleotides, the concentration of polymerase, and, to some extent, the concentration of the primer/template.

In addition, experience has shown that apyrase preparations are more unstable than other components of the pyrosequencing reaction, and that this is clearly a critical component. Moreover, apyrase has been found to bind to secondary structures in the DNA template, leading to loss of activity. This means that apyrase activity may be difficult to control when sequencing certain templates with complex secondary structures. Another disadvantage with apyrase is that it degrades ATP such that the maximum light signal from the luciferase reaction is only transient. Removal of apyrase from the light-generating reaction would result in a plateau of stable light output that can be expected to simplify signal processing and increase sensitivity through increased data sampling.

Thus, it would be preferable to be able to perform the sequencing by synthesis reaction in another way, obviating the need for apyrase or at least reducing or eliminating the drawbacks discussed above. Especially, it would be advantageous if it would be possible to remove apyrase from the environment in which the polymerase reaction is run, thus removing the risk for minus-shift due to excessive nucleotide degradation.

WO94/20831 discloses a micropipette tip that is prepared for chromatography, which allows the immobilisation of DNA in the tip for separation purposes. The immobilising structure in the tip is of fibrous material.

U.S. Pat. No. 5,171,537 discloses an activated, removable micropipette tip comprising a solid phase, which allows binding of various biomolecules, such as proteins and nucleic acids, for use in various diagnostic analysis, such as PCR.

Thus, it is known that the prior art includes the use of disposable devices, such as a micropipette tip for the immobilisation of biomolecules, such as nucleic acids, for diagnostic purposes.

It is an object of the invention to provide a novel format for performing a sequencing-by-synthesis reaction avoiding the drawbacks mentioned above. Moreover, an object of the invention is to further improve the present pyrosequencing reaction. Another object is to develop the concept of a disposable device, such as a micropipette tip, as a tool for performing biomolecular reactions.

### SUMMARY OF THE INVENTION

These and other objects are achieved by a tubular member, preferably a pipette tip, which allows a sequencing-by-synthesis reaction to be performed in the tubular member. Thus, the tubular member of the invention will act as a reaction chamber for an enzymatic reaction involving a nucleic acid molecule. Also, the need for apyrase is obviated or at least partly reduced and/or removal apyrase to another environment is facilitated, and a completely novel format for performing the Pyrosequencing™ reaction is provided.

Furthermore, the invention is directed to a method for performing a sequencing-by-synthesis reaction, wherein the entire sequencing-by-synthesis reaction is performed within the tubular member, and the detection of the outcome of the reaction is performed on/in the tubular member.

In another aspect the invention is directed to a method for performing the first steps of the sequencing-by-synthesis reaction in the tubular member, whereby the detection of the outcome of the reaction is detected outside the tubular member.

In one embodiment the invention involves using apyrase in the detection step but not in the extension step (thus avoiding minus shift but allowing re-use of the detection reagents).

In a further aspect, means allowing the immobilisation of enzymes used for the detection of the sequencing-by-synthesis reaction are provided, thereby offering a novel way to detect the outcome of the reaction.

In yet another aspect, an automated system for performing the methods of the invention using the tubular members of the invention is provided, thereby allowing an optional integration of DNA-preparation, PCR, and sequencing-by-synthesis into an automated system.

Some of the advantages that are achieved by the invention are for example: (a) The tubular member format allows washing of the template between each reaction step and each reaction cycle, which reduces the accumulation of unincorporated nucleotides and by-products leading to plus-shift, which in turn improves the sequencing quality. (b) Compartmentalisation of individual nucleotide incorporation steps makes it possible to exclude or to reduce the use of apyrase. Removal of apyrase simplifies optimisation of the integrated, homogeneous Pyrosequencing™-reaction for the reasons listed above (no competition between polymerase and apyrase for nucleotides, no dependence on apyrase that can be unstable, or bind to secondary structures in the template). Also, removal of apyrase can increase the amount of light released per nucleotide incorporation and the integration time for signal detection, thus increasing sensitivity. (c) The sample preparation is simplified, since the same format may be used for the template immobilisation, denaturation and annealing of sequencing primer. (d) The reaction format of the present invention is automatable in a conventional robot that can perform all steps of the sequencing-by-synthesis reaction, and also integrate upstream operations including DNA preparation (which can be performed in similar tubular members (pipette tips) with a suitable solid-phase), and PCR (polymerase chain reaction). (e) By splitting the polymerisation part of the sequencing-by-synthesis from the detection part, different conditions can be used, which makes it possible to optimise each part of the reaction. (f) By performing the light-generating reaction in physically separate vessels, the risk of cross-contamination is reduced and also the risk of cross-over of light signal between samples.

The consumption of Pyrosequencing™-reagents may increase by using the format of the invention. However, by compartmentalising different parts of the reaction, so that the part employing the most expensive reagents (conversion of PPi to light) is performed outside the tubular member, the consumption of expensive reagents is reduced. Also, the omission of apyrase may increase the sensitivity of the detection, and thus reduce the reagent consumption. Moreover, by immobilising e.g. sulphurylase and luciferase, the consumption of reagents is further reduced. Also, split reaction means possibility of reducing dNTP concentrations since no competition with apyrase. Yet another advantage according to the invention is that the template is captured in an easy to handle format that permits washing away excess sequencing primer and even recycling a template to examine another region.

An additional phenomenon involves templates with complex secondary structures that disturb the activity of the DNA polymerase, and thus cause incomplete incorporation and minus shift. These errors naturally increase with increasing number of primer extensions. The problem of secondary structures is possible to solve by the present invention, since different parts of the reaction may be compartmentalised, and thus e.g. temperature (which is a critical parameter for the occurrence of secondary structures) may be raised during the extension part of the reaction (using a temperature stable polymerase) and a temperature that is optimal for luciferase (about 25° C.) may be used during the detection part.

Still another advantage with the present invention is that it is possible to run sample preparation and sequencing in the same format.

### BRIEF DESCRIPTION OF THE DRAWINGS

FIG. 1: in situ pyrosequencing reaction with real-time detection. Signals obtained from a primer/template that should give the sequence CT.

FIG. 2: in situ pyrosequencing reaction with real-time detection. Signals obtained from a primer/template that should give the sequence CT. Summary of data from FIG. 1.

FIG. 3: Detection of signal after ejection of reaction mix into a clear plastic well placed over a CCD camera. C1 and T gave signals. The other treatments were controls.

FIG. 4: Detection of signal after ejection of reaction mix into a clear plastic well placed over a CCD camera. C1 and T gave signals. The other treatments were controls. Summary of data in FIG. 3.

FIG. 5: Raw data from detection of PPi in liquid ejected after primer extension in tip. Signals obtained from controls and polymerase reactions with incorrect (G) or correct (C) nucleotide.

FIG. 6: Primer extension in tip and PPi detection in external well. Summary of results obtained from sequencing a template that should give the sequence CTG.

FIG. 7: Analysis of a 57 bp PCR product containing the SNP rs255098, using Pyrosequencing. The sequence is C/TGCTCTGA (SEQ ID NO:1) i.e. a heterozygote at the polymorphic position.

FIG. 8: Sequencing a PCR product with extension in the tip and detection in a separate chamber. Summary of data from 3 experiments. The order of presentation of nucleotides is the same as that in the results from analysis by Pyrosequencing shown in FIG. 7.

FIG. 9: Primer extension in tip with preloaded polymerase and PPi detection in external well. The template/primer was preloaded with DNA polymerase at pH 7.6, washed extensively, and then exposed to nucleotide solutions. Released PPi was detected in a separate well. Summary of results obtained from experiments in triplicate sequencing a template that should give the sequence CTG.

FIG. 10: Primer extension in tip with preloaded polymerase and PPi detection in external well. The template/primer was preloaded with DNA polymerase at pH 6.3, washed extensively, and then exposed to nucleotide solutions. Released PPi was detected in a separate well. Summary of results obtained from experiments in triplicate sequencing a template that should give the sequence CTG.

FIG. 11: Analysis by Pyrosequencing of PCR products obtained from DNA isolated from whole blood as compared to control DNA. Upper panel: Standard DNA; Middle panel: DNA isolated using GFX Genomic Blood DNA Purification Kit; Lower panel: DNA isolated in pipette tip.

FIG. 12: Principal drawing of a detector for detection in a pipette tip according to the invention.

FIG. 13: Principal drawing of detection in a flow cell in a split process according to invention.

FIG. 14: Principal drawing of a detector using a plastic film in a split process according to the invention.

### DEFINITIONS

By a “luminescent biomolecular reaction” is meant a reaction in which the activity of an enzyme, or the level of a compound, is monitored in a light-emitting reaction, whereby the light-emission is an enzymatic reaction (luciferase enzyme). For example, the Pyrosequencing™ reaction is a luminescent biomolecular reaction.

Pyrosequencing™ is a sequencing method developed at the Royal Institute of Technology in Stockholm (Nyren, P. (2001) Method for sequencing DNA based on the detection of the release of pyrophosphate and enzymatic nucleotide degradation; Patent: US-B1-6258568, WO 98/28440; Ronaghi, et al. 1998, Alderbom et al.,2000). The method is based on “sequencing by synthesis” in which, in contrast to conventional Sanger sequencing, the nucleotides are added one by one during the sequencing reaction. The principle of the Pyrosequencing™ reaction is as follows: A single stranded DNA fragment (attached to a solid support), carrying an annealed sequencing primer acts as a template for the Pyrosequencing™ reaction. In the first two dispensations, substrate and enzyme mixes are added to the template. The enzyme mix consists of four different enzymes; DNA polymerase, ATP-sulfurylase, luciferase and apyrase. The nucleotides are sequentially added one by one according to a specified order dependent on the template and determined by the user. If the added nucleotide matches the template, the DNA polymerase incorporates it into the growing DNA strand and PPᵢ is released. The ATP-Sulfurylase converts the PPᵢ into ATP, and the third enzyme, luciferase, transforms the ATP into a light signal. Following these reactions, the fourth enzyme, apyrase, degrades the excess nucleotides and ATPs, and the template is ready for the next reaction cycle, i.e. another nucleotide addition. Since no PPᵢ is released unless a nucleotide is incorporated, a light signal is produced only when the correct nucleotide is incorporated.

By a “template nucleic acid sample” is meant a nucleic acid sample of DNA- or RNA-type that is analysed by the luminescent biomolecular reaction.

By “means for binding nucleic acid molecules” is meant some kind of means, such as solid phase beads, an active membrane, a gel or the like, allowing the immobilisation of a desired sample nucleic acid.

By a “tubular member” is meant a pipette tip, a capillary tube, a piece of tubing or the like. In this disclosure reference will be made to both a tubular member and a pipette tip. However, it is the intention to not limit the invention to a pipette tip (which is one preferred variant of a tubular member).

### DETAILED DESCRIPTION OF THE INVENTION

The basis of the invention is to create a novel format for the sequencing-by-synthesis reaction, especially the Pyrosequencing™ reaction, optionally including upstream sample preparation steps. By performing the principal steps of the reaction in a tubular member, such as a pipette tip, wherein the template nucleic acid sample is immobilised, completely new conditions and opportunities arise.

Accordingly, in a first aspect the invention refers to a tubular member having therethrough an axial bore one end of which is releasably insertable over the nozzle of a pipettor to connect said member to said pipettor, wherein the bore comprises means for binding nucleic acid molecules for a subsequent luminescent biomolecular reaction, wherein the means for binding nucleic acid molecules comprises at least one filter or membrane (made of, for example, polypropylene) positioned in the bore, on which filter or membrane solid-phase beads for binding nucleic acid molecules are positioned. Hereby, a tubular member is provided, which is adapted for the immobilisation of the desired nucleic acid sample. Also, a solid-phase for the immobilisation of nucleic acid molecules is held in position in the pipette tip. The use of two filters separated to trap beads in a volume of 0.1-5 μL allows the possibility of avoiding stray beads clinging to the interior walls of the tip, which therefore is an advantageous option.

Preferably, the tubular member is a pipette tip.

By a “pipette tip” is meant a conventionally used micropipette tip (such as those routinely used in laboratories to dispense volumes in the range 1-1000 μLs, for example as manufactured by Molecular BioProducts, Inc. USA) that is adapted to allow binding of biomolecules.

The invention also refers to other tubular devices, such as a capillary tube, a piece of a tubing, which basically has the same function as a pipette tip, i.e. it is possible to provide it with means for binding nucleic acid molecules, and it is adapted to allow an enzymatic reaction to occur within the device and a light reaction to be detected from the device.

By a “pipettor” is meant a conventionally used pipettor (for example Eppendorf pipettes manufactured by Brinkman Instruments, Inc., USA) or an automatically controlled pipettor robot (for example, Biomek robots manufactured by Beckman Coulter, Inc., USA).

In one embodiment, the tubular member is further adapted to allow the detection of a luminescent biomolecular reaction occurring in the tubular member. Hereby, the light signal that is produced in the Pyrosequencing™ reaction upon nucleotide incorporation is detectable.

Also, the use of filter/membrane and beads can be replaced by the use of an active membrane, such as SAM² Biotin Capture Membrane (Promega Corporation, USA), or a membrane made modified polypropylene (Borcherding et al, 2003), which has the ability to immobilise the desired sample molecules.

In yet another embodiment, the solid-phase beads are streptavidin-coated beads with a binding capacity that is sufficient for capturing 0.01-5 picomoles of biotinylated biomolecule. Hereby, the sample molecule is readily immobilised by the use of a biotin-part on the sample molecule. To provide a biomolecule with a biotin tag is a standard technique for a person skilled in the art. DNA is routinely labelled with biotin by various methods (e.g. Langer et al 1981, Agrawal et al 1986; Sambrook and Russell, 2001) or more commonly synthesised with biotin at their 5′ end (e.g. Zhao and Ackroyd, 1999) or even with internal biotinylation. Proteins can be biotinylated by both chemical and enzymatic means (Sambrook and Russell, 2001; Chapman-Smith and Cronan, 1999). Furthermore, many biomolecules, including DNA, RNA, and certain proteins, are supplied by the manufacturer in a biotinylated form.

In still another embodiment the tubular member has a transparency of at least 10%, more preferably at least 50%, even more preferably at least 80%, and most preferably at least 90%). Hereby, the occurrence of a light signal inside the tubular member is readily detectable outside the member.

Liquids that are used for the different reaction steps can be aspirated from wells that are common for different samples (e.g. buffers and enzyme mixes) or specific wells (e.g. templates and primers). Alternatively, liquids do not necessarily have to be aspirated, but may be pumped into the tubular members from above, e.g. through channels in a robot head.

Also, in one embodiment the invention refers to a tubular member wherein at least one enzyme, such as sulphurylase or luciferase, is immobilised to the solid-phase beads. Hereby, the action of the immobilised enzyme(s) will be controlled in an alternative way. The enzyme is preferably covalently coupled to the solid-phase, more preferably by a streptavidin-biotin coupling.

Moreover, in another embodiment the invention refers to the use of a tubular member of the invention, for immobilising a nucleic acid sample and performing an enzymatically based reaction involving the nucleic acid sample.

By a “nucleic acid sample” is meant a nucleic acid molecule that is of interest to analyse, such as DNA or RNA.

By an “enzymatic reaction involving the nucleic acid sample” is meant a reaction or assay involving submitting the immobilised nucleic acid molecule to the action of an enzyme, thereby achieving a possible change in the structure or properties of the nucleic acid molecule, resulting in a detectable change. For instance such an enzymatic reaction can be a polymerase-catalysed reaction, wherein a nucleotide is added to a complex of a nucleic acid sample molecule and an oligonucleotide primer.

Accordingly, the enzymatically based reaction is a polymerase reaction.

The unit for detecting light from a bioluminescent reaction in a tubular member, the detector unit, comprises a detector which, for example, is chosen from a phototransistor, a photomultiplier or a CCD camera chip, and an optic system (see FIG. 12). The detector and the optic system are built into a housing. A hood around the pipettor/tubular member/pipette tip (e.g. a tubular hood) is preferably used in order to protect the area to be detected from stray, external light. The optic system comprises e.g. a lens and a reflector.

Normally, the area from which light is to be detected is positioned at the level of the means for binding nucleic acid molecules. The detector unit is for example attached to the tubular member, or is a separate unit, whereby the tubular member is moved into the detection unit at the time for detection. If several tubular members are used in parallel, a corresponding number of detection units could be mounted together.

In a second aspect, the invention refers to a method for performing a sequencing by synthesis reaction in a tubular member comprising means for binding nucleic acid molecules as described above, comprising the steps of:
(a) immobilising a template nucleic acid sample on the means for binding nucleic acid molecules, and annealing an oligonucleotide primer to the nucleic acid template, which primer is designed to bind to a predetermined position on the template;
(b) extending the oligonucleotide primer by the action of a polymerase with one nucleotide directly adjacent to its 3+ end, whereby the nucleotide that extends the primer is complementary to the nucleic acid template in this position;
(c) detecting the identity of the nucleotide that extends the primer;
(d) optionally repeating step (b)-(c).

The immobilisation of the template nucleic acid sample to the means for binding nucleic acid molecules and the primer annealing to the template may be performed in any order, i.e. a primer/template complex may be generated outside the tubular member, and is subsequently immobilised to the means for binding nucleic acid molecules, or the template may first be immobilised and the primer is then annealed to the template within the tubular member.

The template nucleic acid sample is a DNA or RNA sample.

In one embodiment, the template nucleic acid sample is prepared by means of optionally isolating and amplifying (by polymerase chain reaction or an alternative, isothermal method) the sample before it is immobilised in the tubular member of step (a).

The template nucleic acid sample is aspirated into the tubular member under conditions allowing immobilisation to the means for binding nucleic acid molecules. Such conditions depend on the method of immobilisation. For example, if the template nucleic acid has been generated using a biotinylated primer then the template nucleic acid can be immobilised on a streptavidin-coated solid-phase at room temperature in the presence of a high-salt buffer (typically pH 7.5 and 1-2M NaCl).

If the immobilised template nucleic acid sample is double-stranded DNA, the sample is treated under denaturing conditions (for example by exposure to high pH in the form of 100-200 mM sodium hydroxide, or high temperature e.g. 80-95° C.) in order to become single-stranded.

Thereafter, washing (by repeated aspiration and ejection, or otherwise flow-through of a suitable buffer, usually of low ionic strength and at approximately pH 7.5) of the template is preferably conducted, in order to remove reagents that are undesired in the subsequent steps.

Thereafter, the oligonucleotide primer is aspirated into the tubular member under conditions (for example, by incubation at 60-80° C. at pH 7.6 in the presence of 2-5 mM Mg²⁺ ions, followed by active or passive cooling to a low temperature, typically room temperature) allowing annealing to the immobilised template nucleic acid sample. In one embodiment, unbound primer is ejected from the tubular member, and optionally the template is washed by repeated aspiration and ejection, or otherwise flow-through of a suitable buffer.

Thereafter, an enzyme mix comprising nucleic acid polymerase, sulphurylase and luciferase, substrate comprising APS and luciferin, and a dNTP is aspirated into the tubular member under conditions allowing extension of the oligonucleotide primer in case the chosen dNTP is complementary to the template in the position directly adjacent to the 3′-end of the primer. Hereby, the use of apyrase, as in a normal Pyrosequencing™ reaction is avoided. However, in one embodiment apyrase may be included in a washing step in order to further reduce the amount of unincorporated nucleotides. Also, by-products are not allowed to accumulate. The risk for a positive shift is reduced and thus a longer stretch of the template can be analysed.

Regarding concentration ranges for the various enzymes and reagents that are used in the methods of the invention the following values may be used (based on the use of a Klenow polymerase. For another polymerase, the values may differ to some extent, especially the dNTP concentration): 10-500, preferably about 100 U/ml Klenow exo-DNA polymerase, 0.1-2.5, preferably about 0.5 U/ml ATP sulphurylase, 1-50, preferably about 10.5 μg/ml luciferase, 0.01-0.5, preferably about 0.15 mg/ml luciferin, 1-20, preferably about 4 μM APS, 0-1, preferably about 0.1 mM EDTA, 0-1, preferably about 0.1 mM DTT and 0.1-10, preferably about 7 μM dATPalpha S or 0.1-10, preferably about 1.8 μM dCTP, or 0.1-10, preferably about 1.2 μMdGTP or 0.1-10, preferably about 2.2 μM dTTP.

The polymerase enzyme is suitably lacking in 3′-5′ exonuclease activity and can be selected from DNA-dependent polymerases, or modified enzymes, such as Klenow exo-(Stratagene), Sequenase, Thermo Sequenase, Thermo Sequenase II (Amersham Biosciences), rTth DNA polymerase (Applied Biosystems), Tli DNA polymerase exo- (Vent (exo-) DNA Polymerase, New England Biolabs), Deep Vent (exo-) DNA Polymerase (New England Biolabs), AmpliTaq DNA polymerase (Amersham Biosciences), Bst DNA Polymerase (New England Biolabs), DyNAzyme I and II DNA Polymerases (Finnzymes Oy); or RNA-dependent polymerases such as HIV-1 RT, M-MuLV RT, AMV RT, RAV2 RT, Thermoscript AMV RT, Superscript II M-MuLV RT, or Tth DNA polymerase.

The dNTP is preferably any one of the four DNA nucleotides dATP, dCTP, dTTP or dGTP. For the analysis of DNA and RNA dUTP is preferably used instead of dTTP. Also, modified variants of these natural nucleotides may be used (e.g. dITP, 7-deaza-2′-deoxyguanosine 5′-triphosphate, 2′-deoxyadenosine-5′-O′-(1-thiotriphosphate)). The order in which the nucleotides are added varies depending on the sequencing strategy. If the positions in the sample nucleic acid that are to be identified are known in advance, the addition of dNTP may be chosen in order to reduce the number of dNTP-additions, i.e. to maximise the number of nucleotide additions resulting in an incorporation of nucleotide. Other strategies may however be chosen, for instance, the various dNTP:s may be added in a cyclic fashion, i.e. starting with e.g. dATP, then adding e.g. dCTP, dGTP and dTTP and then starting all over again with dATP and so on.

Thereafter, the occurrence of a primer extension is detected by the monitoring of a light signal, whereby the light detector is positioned above the tubular member or on the side of the tubular member, wherein the light signal is detected from inside the tubular member, or whereby the light detector is positioned below the tubular member, wherein the light signal is detected after ejecting at least a part of the reaction mixture. Hereby, a light signal resulting from the bioluminescent reaction, such as the Pyrosequencing™-reaction, is readily detected.

There are many methods available for the detection of light as a result of a luminescence reaction, such as detection on photographic films, x-ray films, by the use of a photomultiplier tube (PMT), charge coupled device (CCD) camera or phototransistor. For the present invention the use of luminometric detection by PMT or CCD camera is preferable, since such methods allow for a quantitative measurement of light emission.

Steps (c)-(d) are then optionally repeated e.g. 1 to 2000 times, for example 1-500 or 10-100 times, depending on the analysis that is performed. Preferably, the template is washed between each cycle by repeated aspiration and ejection, or otherwise flow-through of a suitable buffer, usually of low ionic strength and at approximately pH 7.5.

In a third aspect, the invention is directed to a method for at least partly performing a sequencing by synthesis reaction in a tubular member comprising means for binding nucleic acid molecules as described above, comprising the steps of:
(a) immobilising a template nucleic acid sample on the means for binding nucleic acid molecules, and annealing an oligonucleotide primer to the nucleic acid template, which primer is designed to bind to a predetermined position on the template;
(b) extending the oligonucleotide primer by the action of a polymerase with one nucleotide directly adjacent to its 3′ end, whereby the nucleotide that extends the primer is complementary to the nucleic acid template in this position;
(c) dispensing at least part of the reaction mixture resulting from step (c) into a means for detection;
(d) detecting the identity of the nucleotide that extends the primer in the means for detection;
(e) optionally repeating step (b)-(d).

The immobilisation of the template nucleic acid sample to the means for binding nucleic acid molecules and the primer annealing to the template may be performed in any order, i.e. a primer/template complex may be generated outside the tubular member, which is subsequently immobilised to the means for binding nucleic acid molecules, or the template may first be immobilised and then the primer is annealed to the template within the tubular member.

Hereby, by splitting the entire sequencing-by-synthesis reaction in two major parts, wherein one, the extension part, is performed in the tubular member, and the second, the detection part, is performed outside the tubular member, several advantages may be achieved. In the extension phase, PPi is produced (in the event of a nucleotide incorporation) and in the detection phase the presence of PPi is determined and quantified. For instance, different conditions, such as different temperatures, pH, salt concentration, substrate concentration, may to a greater extent be used for the different parts of the reaction. For example, this would permit the use of thermostable DNA polymerases, whilst not necessitating the development of thermostable enzyme cascade systems for PPi detection. Normally, the optimal temperature for the enzyme system that is used to convert PPi to light is about 25-28° C. This approach also provides means to include apyrase in the detection step, thereby permitting re-use of the detection mix (i.e. the reagents that are used in the detection step), but removing the competition between apyrase and polymerase in the extension step and thereby reducing the risk of minus shift. Splitting the reaction is especially an advantage when sequencing RNA, since secondary structures can be avoided by using higher temperatures. Further, for RNA sequencing, the thermostable reverse transcriptase is the normative polymerase. Moreover, in RNA sequencing degradation of the RNA template is a key problem, and this is more easily controlled in an environment that is restricted to the reverse transcriptase step. Also, the optical qualities of the tubular member are not relevant, since the detection is performed outside the tubular member.

Thus, in one embodiment step (b) (primer extension) is performed at a temperature that is optimal for the polymerase enzyme, i.e. about 37-72° C., depending on the polymerase that is used, and step (d) (PPi detection) is performed at a temperature that is optimal for luciferase, i.e. about 25-28° C.

The template nucleic acid sample is a DNA or RNA sample.

In one embodiment, the template nucleic acid sample is prepared by means of optionally isolating and amplifying (by polymerase chain reaction or an alternative, isothermal method) the sample before it is immobilised in the tubular member of step (a).

The template nucleic acid sample is aspirated into the tubular member under conditions allowing immobilisation to the means for binding nucleic acid molecules.

If the immobilised template nucleic acid sample is double-stranded DNA, the sample is treated under denaturing conditions, in order to become single-stranded.

Thereafter, the oligonucleotide primer is aspirated into the tubular member under conditions allowing annealing to the immobilised template nucleic acid sample. In one embodiment, unbound primer is ejected from the tubular member, and optionally the template is washed (see above).

As a next step, nucleic acid polymerase, and a dNTP is aspirated into the tubular member under conditions allowing extension of the oligonucleotide primer in case the chosen dNTP is complementary to the template in the position directly adjacent to the 3′-end of the primer. Hereby, in case of an incorporation, pyrophosphate (PPᵢ) will be released. If no incorporation occurs, essentially no pyrophosphate will be present in the reaction mixture.

Thereafter, at least a part of the reaction mixture in the tubular member will be dispensed to the means for detection. Thus, the part of the reaction mixture that is dispensed to the means for detection will comprise pyrophosphate if an incorporation has occurred in the tubular member. If no incorporation has occurred, essentially no pyrophosphate will be present in the means for detection. Therefore, the potential presence of pyrophosphate can be used for determining the outcome of the extension reaction.

Steps (b)-(d) are then optionally repeated. Preferably, the template is washed between each cycle.

Moreover, in one embodiment step (b)-(c) are repeated and step (d), the detection, is performed at a separate time. Hereby, a plurality of dispensed extension reaction products is collected, before respective dispensed extension reaction product is analysed for the occurrence of pyrophosphate. This is one way of further simplifying the reaction. Thus it is possible to measure the product of one incorporation whilst the next nucleotide is incorporated into the primer/template-complex.

In one embodiment, the means for detection is a container, such as a well in a microtiter plate, comprising a detection solution comprising sulphurylase, luciferase, APS and luciferin, whereby the dispensed reaction mixture of step (b) and the detection solution are mixed, thereby allowing a light signal to develop in case of a primer extension, which light signal is detected from within the container. The container may be adapted for single or multi-use. In the multi-use case, the container may be used for detection until a resolution limit has been reached. Alternatively, apyrase may be included in the detection solution in order to eliminate excess ATP and dNTPs after the initial light signal. These modifications mean that preferably only a small volume is dispensed from the tubular member for each detection in order to make it possible to use the multi-use detection container for as many detection cycles as possible.

In another embodiment, the means for detection is a flow cell (see example 7), comprising a detection solution comprising sulphurylase, luciferase, APS and luciferin, whereby the dispensed reaction mixture of step (b) and the detection solution are mixed, thereby allowing a light signal to develop in case of a primer extension, which light signal is detected from within the flow cell. Hereby, the occurrence and identity of a nucleotide incorporation in the previous primer extension step is determined.

In another embodiment the means for detection is a film or a well (see example 8), comprising freeze-dried detection reagents comprising sulphurylase, luciferase, APS and luciferin, and whereby the dispensed reaction mixture of step (b) and the freeze-dried detection reagents are mixed, thereby allowing a light signal to develop in case of a primer extension, which light signal is detected from the film or well.

In still another embodiment, the means for detection is a film or a well, comprising a detection solution comprising sulphurylase, luciferase, APS and luciferin, whereby the dispensed reaction mixture of step (b) and the detection solution are mixed, thereby allowing a light signal to develop in case of a primer extension, which light signal is detected from the film or well.

In yet another embodiment the means for detection comprises a capillary detection chamber, comprising freeze-dried detection reagents comprising sulphurylase, luciferase, APS and luciferin, and whereby all or a part of the dispensed reaction mixture of step (b) is absorbed into the capillary detection chamber by capillary forces, whereby the dispensed reaction mixture of step (b) and the freeze-dried detection reagents are mixed, thereby allowing a light signal to develop in case of a primer extension, which light signal is detected from the capillary detection chamber. Hereby, the volume of the dispensed reaction mixture of step (b) that is subjected to detection is easily controlled. Also, an arrangement according to this embodiment reduces problems of evaporation during detection.

In still another embodiment the enzymes sulphurylase and/or luciferase is (are) immobilised, for example through biotinylation of the enzyme followed by immobilisation on a streptavidin-coated solid-phase, in the means for detection, and whereby the dispensed reaction mixture of step (b) is mixed with APS, luciferin and optionally sulphurylase and/or luciferase, and is allowed to flow through the immobilised enzyme(s), thereby allowing a light signal to develop in case of a primer extension, which light signal is detected from the means for detection. Hereby, the amount of reagents that is used can be reduced, which will reduce the overall costs for the reaction. Normally, luciferase and luciferin are considered to be the most expensive reagents, and the immobilisation of luciferase would thus reduce costs significantly. Also, the light production can advantageously be limited through the activity of the immobilised enzymes (sulphurylase and/or luciferase), thereby minimising variation in signal between incorporations. An alternative is to carefully dispense or flow-control luciferin and/or APS.

Consequently, in a fourth aspect the invention refers to a means for detection of a bioluminescent reaction as disclosed above according to the third aspect of the invention, having immobilised thereto at least one of the enzymes luciferase and/or sulphurylase. These proteins may be immobilised through biotinylation and capture on a streptavidin-coated solid-phase (see Sambrook and Russell, 2001) or by other means, such as covalent attachment (Immobilization of Enzymes and Cells, Methods in Biotechnology Series, ed. G. Bickerstaff, Humana Press, 1996). Hereby, a means for detection, such as a flow cell, or any one of the ether embodiments described in the third aspect of the invention, is provided which is used in the method of the invention.

In a fifth aspect, the invention refers to an automated system for performing the method as described above, comprising at least one pipettor (and for example 1, 2, 4, 8, 96, 384 or 1536 pipettors or pipettor formats) allowing at least one tubular member, preferably a pipette tip, as described above to be releasably insertable over the nozzle of the pipettor to connect said tubular member to said pipettor, and means for controlling the system. Hereby, the steps of the methods of the invention, as well as optionally preparation and amplification steps, may be automated. Also, several samples may be analysed in parallel fashion, thereby increasing the throughput of samples. The means for controlling the system may for example be a computer program, which is capable of controlling the mechanical parts of the system in an automated fashion.

In still another aspect the invention refers to a computer program for causing a computerised apparatus to determine the identity of at least one nucleotide in a nucleic acid molecule, by way of sequentially detetcing a measurable quantity of a reaction when the nucleotide is incorporated to form a base pair with the complementary base in the nucleic acid molecule, comprising a computer readable code means which when run causes the computerised apparatus to perform the method steps of the invention.

Also, the invention refers to a computer program product for causing a computerised apparatus to determine the identity of at least one nucleotide in a nucleic acid molecule, by way of sequentially detecting a measurable quantitiy of a reaction when the nucleotide is incorporated to form a base pair with the complementary base in the nucleic acid molecule, comprising a computer readable medium, and a computer program as disclosed above, the computer program being recorded on said computer readable medium.

In one embodiment, the computer readable medium comprises a memory chip.

A well specific dispensing of reagents for the tubular member-based reaction according to the invention, can be achieved as follows: a linear arrangement of tubular members (for example 8) are held stationary (only moved up and down for aspiration or ejection) during the aspiration steps, whilst strips with wells containing the reagents (for a DNA analysis) polymerase and dATP, polymerase and dCTP, polymerase and dGTP and polymerase and dTTP, and PPi-detection reagents (APS, luciferin, luciferase and sulphurylase) are moved independently in the horizontal plane under each tubular member in order to provide the correct dNTP depending on the individual sample that is immobilised in the tubular member. After receiving the PPi-detection reagents all tubular members are moved to the stationary detector wells into which they are inserted for one second to several minutes, to measure the light output.

DNA isolation can be performed using a suitable solid-phase (e.g. silica) that is immobilised in a tubular member (preferably a pipette tip) of practically the same kind as the tubular members of the invention.

In a sixth aspect of the invention, a tubular member (preferably a pipette tip) is used in order to purify the nucleic acid sample, e.g. in the form of genomic DNA. This can be achieved in a method comprising the following steps:

mixing a sample, such as whole blood (preferably 0.1-10 μL), with extraction buffer designed to release cellular DNA and with a composition that promotes binding of DNA to silica-based solid-phases (for example, containing a chaotropic agent, such as guanadinium isothiocyanate);
(a) aspirating the mix into the tubular member containing e.g. a silica-based matrix, thereby immobilising nucleic acid sample;
(b) ejecting unbound material;
(c) washing the immobilised sample with washing buffer suitably containing a low salt buffer and 60-80% ethanol;
(d) aspirating elution buffer (for example, water at 70° C.);
(e) ejecting purified nucleic acid sample into means for performing PCR, such as a PCR microtiter plate;
(f) performing the PCR reaction according to an optimised standardised protocol; and
(g) subsequently using the purified nucleic acid sample PCR product in a method according to the above described aspects.

Hereby, the template nucleic acid sample that is analysed in a subsequent sequencing reaction can be isolated by a tubular member, such as a tubular member of the invention.

Step (a) is for example perfomed by using a standard tip to add extraction buffer to the blood sample in a well on a microtiter plate.

After isolation of the sample, it is optionally subjected to amplification by means of e.g. PCR. Thereafter, the amplified sample may be analysed by the methods of sequencing according to the invention.

The entire chain of steps from isolation of the sample nucleic acid to the detection of incorporated nucleotide in the sample template can in one embodiment be integrated in e.g. two modules in an automated system. For instance, in a preferred embodiment a pre-PCR module and a post-PCR module are used, wherein in each module a tubular member-based mode, as outlined above, is used. In the system, means for heating and detection is also included.

In yet another aspect, the present invention refers to a kit for use in any one of the methods according to the aspects described above, comprising a tubular member, preferably a pipette tip, according to the invention and reagents for performing primer extension and/or reagents for performing PPi detection.

By reagents for performing primer extension are meant a suitable polymerase enzyme, dNTP, and optionally other reagents. By reagents for performing PPi-detection are meant sulphurylase, luciferase, luciferin and APS, and optionally other reagents. Optionally, apyrase may also be included in the kit.

In still another aspect, the invention refers to a kit for use in any one of the methods according to the aspects described above, comprising a tubular member, preferably a pipette tip, according to the invention for at least partly performing a sequencing-by-synthesis reaction, and a tubular member, preferably a pipette tip, adapted for purifying the template nucleic acid sample to be used in a subsequent sequencing-by-synthesis reaction, and optionally reagents for performing primer extension and/or reagents for performing PPi detection.

Regarding optimal temperatures for the reaction steps of the various aspects above, different temperatures are optimal for polymerase and luciferase. For polymerase, about 37° C. is optimal for polymerase of Klenow type. About 50-60° C. is a suitable temperature for a temperature stable polymerase, however up to about 72° C. may be used for a temperature stable Taq polymerase. The optimal temperature for the polymerase is however decided by the characteristics of the enzyme used, and thus the manufacturer's instructions may be referred to for information on this point. Secondary structure problems are however avoided at a higher temperature. For luciferase about 25-37, preferably 25-28° C., or more preferably about 25° C. is a suitable temperature. By using the split-reaction principle of the invention, different temperatures for luciferase and polymerase may be used.

In another embodiment, related to all aspects disclosed above, the immobilised template/primer complex is exposed to polymerase before primer extension is initiated, which polymerase binds to the template/primer complex and remains bound for the remaining cycles of the sequencing reaction. All reaction mixes that are used in subsequent steps thus comprise all necessary components with the exception of the polymerase.

One advantage of pre-loading with polymerase is that enzyme consumption can be expected to be lower than that experienced when polymerase is included in every extension mix containing nucleotide. The ability of a polymerase to remain on the template/primer after the extension step is known as ‘processivity’. A publication, Eckert, K A and Kunkel T A, 1993) indicates that processivity of Klenow exo- DNA polymerase increases on lowering reaction pH from 7.6 to 6.2, but with an accompanying reduction in Kcat (reaction rate). Thus it can be expected that the conditions optimal for high processivity might be different to the normal reaction conditions (which might also coincide with conditions for PPi detection e.g. pH 7.6). In this case, the ability to perform split reactions could be advantageous. In the example here, the polymerase step was run at pH 6.3 and the detection step at pH 7.6, after pH adjustment. This shows the flexibility of the split-reaction. Also, an increased degree of completeness of C-incorporation could be observed.

In one further embodiment related to all the aspects disclosed above, apyrase is used. What is important is that the use of apyrase in combination with polymerase is limited, for the reasons discussed above. For instance, apyrase may be included in a washing step between each reaction cycle, whereby the pipette tip according to the invention is washed with a washing buffer comprising apyrase in order to remove excess of e.g. dNTP. Also, apyrase may be present in a detection container, in which case the light detection reaction is performed outside the tubular member. In yet another variant, apyrase is immobilised to the solid phase (i.e. means for binding nucleic acid molecules within the pipette tip), making it possible to more easily control the effects of apyrase, compared to having apyrase present free in solution.

Moreover, in another embodiment of the invention a fusion protein comprising SSB (single-stranded binding protein) and luciferase, or functional analogues of SSB and/or luciferase, is used in order to bind luciferase locally to the nucleic acid sample template (see Ehn, 2003). Hereby, the action of luciferase is more easily controlled. Also, other variants of immobilised enzymes are fully possible and are included in the scope of the invention.

Now the invention will be described in more detail with regard to the appended examples. These examples should not be construed as limiting the invention in any way, but merely as an illustration of the inventive features. All patents, patent applications, and references cited anywhere in this specification are hereby incorporated by reference in their entirety.

### EXAMPLES

### Example 1

In situ Pyrosequencing Reaction with Real-time Detection

Oligonucleotides used were as follows:

(SEQ ID NO:2)

| | |
|---|---|
| NUSPT: | gtaaaacgacggccagtctgacgaattccagc |

(SEQ ID NO:3)

| | |
|---|---|
| E3PN20b | caacattttgctgccggtcagactgcttaaggtcg-biotin |

The expected sequence from this primer/template combination is shown underlined.

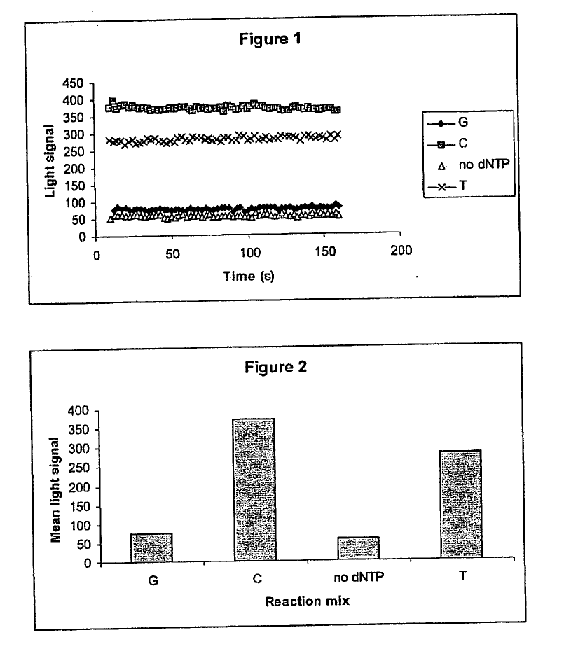

The primer, NUSPT, was annealed to the template, E3PN20b by mixing 6 pmoles of primer with 2 pmoles of template in 10 μL Annealing Buffer (20 mM Tris-acetate, 5 mM MgAc₂, pH 7.6) and incubating for 5 minutes at 80° C. followed by cooling to room temperature. An additional 20 μL of Annealing Buffer was added together with 30 μL of Binding Buffer (10 mM Tris-HCl, 2 M NaCl, 1 mM EDTA, 0.1% Tween-20). AffiniTip™ Step 20 (containing a filter with immobilised streptavidin; Hydros, Inc. USA) was prepared by washing 5 times with 200 μL Annealing Buffer. The template/primer complex was then captured on an AffiniTip by repeated aspiration and ejection over a 5 minute period, using a Eppendorf reference 200 pipette, at room temperature. Unbound material was removed from the filter in the tip by washing with Annealing Buffer. The tip was fixed over a CCD camera such that the region immediately above the filter could be monitored. Reaction mixes, in a volume of 110 μL, were then aspirated into the filter using a 200 μL pipette. The reaction mixes contained 100 U/mL Klenow exo- DNA polymerase, 0.5 U/mL ATP sulphurylase, 10.5 μg/mL luciferase, 0.15 mg/mL luciferin, 4 μM APS, 0.1 mM EDTA, 0.1 mM DTT and 7 μM of dATPalphaS or 1.8 μM dCTP, or 1.2 μM dGTP or 2.2 μM dTTP, depending on the base to be detected, in Annealing Buffer. A control was included that contained no dNTP at all. The signal was monitored over a period of 3 minutes before ejecting the reaction mix. The filter was then washed three times with 200 μL Annealing Buffer before introducing the next reaction mixture. The results of the real-time detection of light indicated a relatively stable level over time (FIG. 1), with a summary of the signals for the sequence in FIG. 2. The correct sequence is CT. Therefore there should be no signal for the G or ‘no dNTP’ controls, as observed here.

### Example 2

Detection of Signal After Election of Reaction Mix The primer NUSPT was annealed to the template E3PN20b, and immobilised on an AffiniTip™ Strep 20 as described in Example 1. The reaction mix, in a volume of 50 μL was then aspirated into the tip, passed through the filter by pulsing with the pipette for 20 seconds before being ejected into a clear plastic well placed above the CCD camera. The order of reaction mixes was as follows:

| | |
|---|---|
| 01 | No dNTP, to test for a stable baseline |
| G | incorrect dNTP, no incorporation expected |
| C1 | correct dNTP |
| C2 | correct dNTP, no incorporation expected if C1 gave |
| | complete extension |
| 02 | no dNTP, to test for a stable baseline |
| T | correct dNTP |

The signal was then monitored for 1 minute. The filter was washed between reaction mixes as in Example 1.

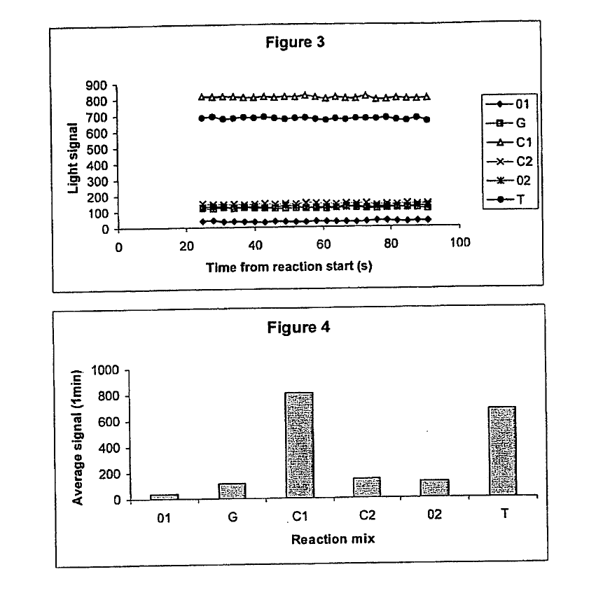

The results are shown in FIG. 3 (real-time measurement) and FIG. 4 (mean light output). Again, stable signals were obtained and the expected signals were obtained for correct dNTPs (C and T) and controls (01, G, C2 and 02).

### Example 3

Primer Extension in Tip and PPi Detection in External Well; Including Steps for Preparation of Single-stranded DNA (NT060:5 p 27)

Oligonucleotides used were as follows:

(SEQ ID NO:2)

| | |
|---|---|
| NUSPT: | gtaaaacgacggccagtctgacgaattccagc |

(SEQ ID NO: 3)

| | |
|---|---|
| E3PN20b | caacattttgctgccggtcagactgcttaaggtcg-biotin |

The expected sequence from this primer/template combination is shown underlined. AffiniTip™ Strep 20 was prepared by washing 5 times with 200 μL Annealing Buffer. The biotinylated template E3PN19b was bound to the filter in the AffiniTip by aspirating and dispensing several times 4 pmoles of E3PN20b in 30 μL Annealing Buffer and 30 μL Binding Buffer, for 5 minutes at room temperature. The bound oligonucleotide was then exposed to Denaturing Solution (0.2 M NaOH), which is designed to remove the non-bound strand of double-stranded DNA, for 2 minutes, followed by washing once with Denaturing Solution, 5 times with Washing Buffer (20 mM Tris-acetate, pH 7.6), and 3 times with Annealing Buffer. The AffiniTip was then prepared for annealing the primer by aspirating 55 μL Annealing Buffer into the filter and incubating at 80° C for 2 minutes. This solution was then replaced with 55 μL Annealing Buffer containing 30 pmoles of NUSPT, preheated to 80° C., and the AffiniTip was incubated for a further 1 minute before being allowed to cool to room temperature. The Annealing Buffer and excess primer were ejected and the filter was washed 3 times with 200 μL Annealing Buffer. The primer/template complex was then exposed to reaction mixes containing DNA polymerase together with one of the dNTPs by aspirating 55 μL of the reaction mix (5U Klenow exo- DNA polymerase and 1.5 μM dCTP or 1 μM dGTP or 1.8 μM dTTP or no dNTP) and incubating for 1 minute at room temperature.

The order of reaction mixes was as follows:

| | |
|---|---|
| AB | buffer, to test for a stable baseline |
| G | incorrect dNTP, no incorporation expected |
| C1 | correct dNTP |
| C2 | correct dNTP, no incorporation expected if C1 |
| | gave complete extension |
| T | correct dNTP |
| G | correct dNTP |

A 45 μL aliquot of reaction mix was transferred to a PSQ96 Plate and placed in a PSQ96 Pyrosequencing Instrument for detection of PPi. The instrument was used to dispense Enzyme and Substrate mixes to give a final volume in the well of 55 μL with the composition 0.5 U/mL ATP sulphurylase, 10.5 μg/mL luciferase, 0.15 mg/mL luciferin, 4 μM APS, 0.1 mM EDTA and 0.1 mM DTT. The light produced was measured using the CCD camera in the instrument.

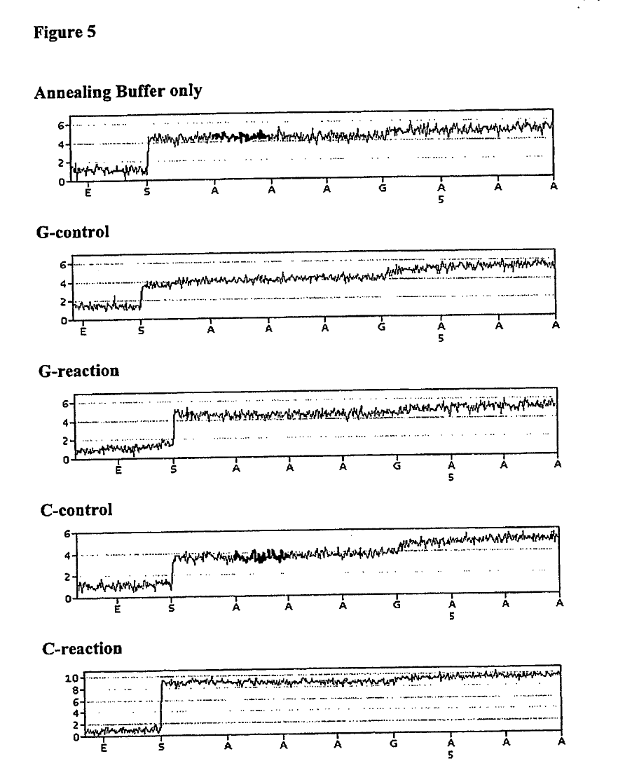

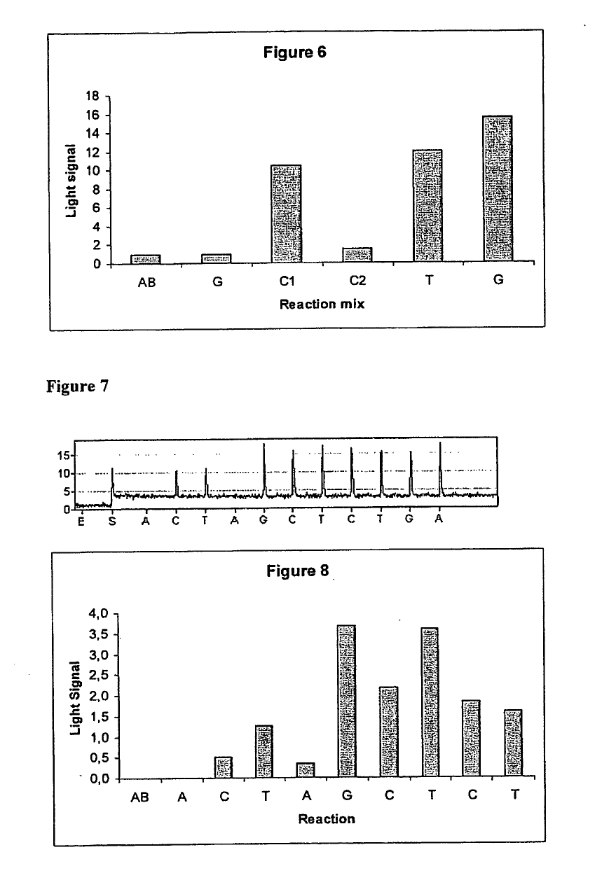

Examples of the output from the PSQ96 instrument are shown in FIG. 5. Note that the only well that gave a significant signal over background was that containing the PPi generated from the exposure of the primer/template to the correct base, C. A summary of the signals from the different bases is shown in FIG. 6.

### Example 4

Sequencing a PCR Product with Extension in the Tip and Detection in a Separate Chamber.

Primers and sequence:

B082FP - >
tcagcagacc catagccttc tccctgcctt cctctgccct ccctccagca
ggtggcacag acccttccct gtctcccttg gcggatggtc ccYgctc tga
gtgcagcgga tagggtccca B084FS - >
<- B083RPB with 5′ biotin

A 57 bp PCR product containing the SNP rs255098 was prepared as follows. The PCR mix contained 3 pmoles each of the primers B082FP and B083RPB, 200 μM of dATP, dCTP, dGTP and dTTP, 1.5 mM MgCl₂, 3 ng DNA (prepared from human lymphocytes, Coriell Cell Repositeries at Coriell Institute for Medical Research, USA), and 0.5 U AmpliTaq Gold (Applied Biosystems) in 1×PCR Buffer II (supplied with the enzyme) in a total volume of 15 μL. The mix was subject to thermocycling with the program: 5 min, 95° C.; (15s, 95° C.; 30s, 58° C.; 15s, 72° C.)×45; 5 min, 72° C.

The PCR product was prepared for sequencing on the Pyrosequencing Instrument PSQ96 according to manufacturers instructions. PCR product in 15 μL reaction mix was captured on Streptavidin Sepharose (Amersham Biosciences) and denatured using the Vacuum Prep Tool (Pyrosequencing AB), the sequencing primer B084FS was annealed, and the PCR product was sequenced in a PSQ96 (Pyrosequencing AB). A Pyrogram is shown in FIG. 7 and indicated that the DNA sample was heterozygous in the polymorphic position (C/T).

AffiniTip™ Strep 20 was prepared by washing 5 times with 200 μL Annealing Buffer. The PCR product was then captured, in triplicate, onto AffiniTip™ Strep 20 and sequenced as follows. Forty microlitres, corresponding to 2 pmole PCR product was mixed with 40 μL Binding Buffer. This mixture was aspirated into the AffiniTip and passed through the filter several times during a period of 5 minutes at room temperature. The excess liquid was ejected and 100 μL Denaturing Solution (0.2M NaOH) was aspirated into the filter and left for 1 minute. The filter was then washed once with 100 μL Denaturing Solution, five times with 200 μL Washing Buffer (20 mM Tris-acetate, pH 7.6), and 3 times with 200 μL Annealing Buffer. The AffiniTip was then prepared for annealing the primer by aspirating 55 μL Annealing Buffer into the filter and incubating at 80° C. for 2 minutes. This solution was then replaced with 55 μL Annealing Buffer containing 10 pmoles of sequencing primer B084FS, preheated to 80° C., and the AffiniTip was incubated for a further 1 minute before being allowed to cool to room temperature. The Annealing Buffer and excess primer was ejected and the filter was washed 3 times with 200 μL Annealing Buffer. The primer/template complex was then exposed to reaction mixes containing DNA polymerase together with a dNTP by aspirating 55 μL of the reaction mix (5U Klenow exo- DNA polymerase and 1.5 μM dCTP or 1 μM dGTP or 1.8 μM dTTP or no dNTP) and incubating for 1 minute at room temperature.

The reaction mixes were:
1. Annealing Buffer only—to check the baseline
2. A—incorrect base
3. C—correct base (should give a half signal since the sample is heterozygous)
4. T—correct base (should give a half signal since the sample is heterozygous)
5. A—incorrect base
6. G—correct base (should give a full signal)
7. C—correct base (should give a full signal)
8. T—correct base (should give a full signal)
9. C—correct base (should give a full signal)
10. T—correct base (should give a full signal)

A 45 μL aliquot of reaction mix was transferred to a PSQ96 Plate and placed in a PSQ96 Pyrosequencing Instrument for detection of PPi, including suitable controls for background determination. The instrument was used to dispense Enzyme and Substrate mixes to give a final volume in the well of 55 μL with the composition 0.5 U/mL ATP sulphurylase, 10.5 μg/mL luciferase, 0.15 mg/mL luciferin, 4 μM APS, 0.1 mM EDTA and 0.1 mM DTT. The light produced was measured using the CCD camera in the instrument. The results of the sequencing, as mean values for triplicate measurements, are shown in FIG. 8. The results indicate that the heterozygous positions C and T gave low but measurable signals whilst the following sequence gave higher signals, similar to the pattern in the Pyrogram in FIG. 7. The unevenness of the pattern in FIG. 8 can no doubt be overcome by optimisation of the reactions.

### Example 5

Preloading Immobilised Primer/template with DNA Polymerase

Oligonucleotides used were as follows:

(SEQ ID NO:2)

| | |
|---|---|
| NUSPT: | gtaaaacgacggccagtctgacgaattccagc |

(SEQ ID NO:3)

| | |
|---|---|
| E3PN20b | caacattttgctgccggtcagactgcttaaggtcg-biotin |

The expected sequence from this primer/template combination is shown underlined.

The primer, NUSPT, was annealed to the template, E3PN20b by mixing 12 pmoles of primer with 4 pmoles of template in 10 μL Annealing Buffer (20 mM Tris-acetate, 5 mM MgAc₂, pH 7.6) and incubating for 5 minutes at 80° C. followed by cooling to room temperature. An additional 20 μL of Annealing Buffer was added together with 30 μL of Binding Buffer (10 mM Tris- HCl, 2 M NaCl, 1 mM EDTA, 0.1% Tween-20). AffiniTip™ Strep 20 (containing a filter with immobilised streptavidin; Hydros, Inc. USA) was prepared by washing 5 times with 200 μL Annealing Buffer. The template/primer complex was then captured on an AffiniTip by repeated aspiration and ejection over a 5-minute period, using a Eppendorf Research 300multichannel pipette, at room temperature. Unbound material was removed from the filter in the tip by washing with 5 times with 200 μL Annealing Buffer followed by one wash with 200 μL of Annealing Buffer or MES Buffer (containing 20 mM MES, 5 mM Magnesium acetate, pH 6.3). The immobilised template/primer was then loaded with DNA polymerase by aspirating 50 μL of Polymerase mix containing 25U Klenow exo-DNA polymerase in Annealing Buffer or MES Buffer. The mix was passed repeatedly through the filter by aspirating and ejecting using a multichannel pipette. Excess, unbound polymerase was removed by washing 5 times with 200 μL of buffer only.

The primer/template complex was then exposed to solutions containing one of the dNTPs by aspirating 25 μL of the dNTP solution (3.6 μM dCTP or 2.4 μM dGTP or 4.4 μM dTTP in either Annealing Buffer or MES Buffer) and incubating for 1 minute at room temperature. The mix was then ejected back into the well and the AffiniTip was washed three times with 200 μL of the relevant buffer (AB pH 7.6 or MES pH 6.3) before exposure to the next dNTP solution.

The order of dNTP solutions was as follows:

Buffer to test for a stable baseline

| | |
|---|---|
| G | incorrect dNTP, no incorporation expected |
| C1 | correct dNTP |
| C2 | correct dNTP, no incorporation expected if C1 gave |
| | complete extension |
| T | correct dNTP |
| G | correct dNTP |

A 10 μL aliquot of the reacted dNTP solutions was transferred to a PSQ96 Plate containing 10 μL of the alternative reaction buffer (Annealing Buffer or MES Buffer), and 30 μL of pH-adjusting buffer containing 70 mM Tris-acetate and 2 mM Magnesium acetate, pH 7.6. The plate was placed in a PSQ96 Pyrosequencing Instrument for detection of PPi. The instrument was used to dispense Enzyme and Substrate mixes to give a final volume in the well of 60 μL with the composition 0.5 U/mL ATP sulphurylase, 10.5 μg/mL luciferase, 0.15 mg/mL luciferin, 4 μM APS, 0.1 mM EDTA and 0.1 mM DTT. The light produced was measured using the CCD camera in the instrument.

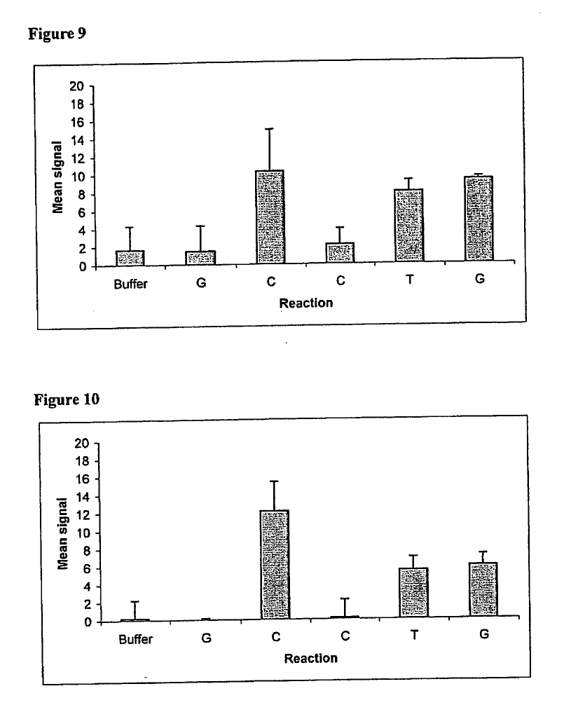

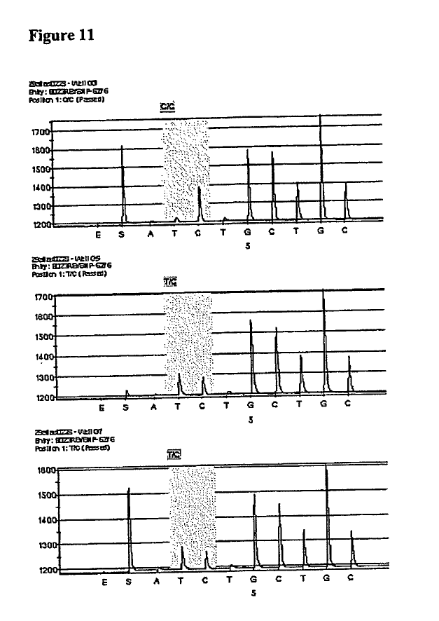

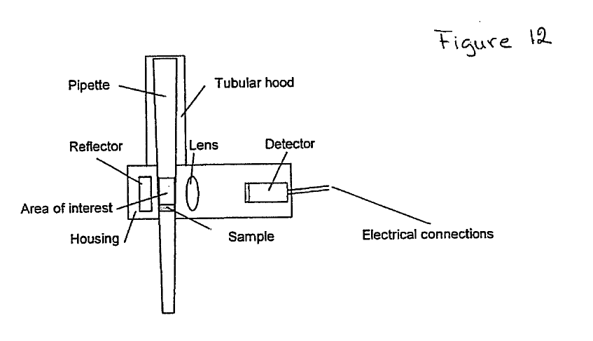

The results for the two buffer systems are shown in FIGS. 9 and 10. The results clearly show that the correct signals can be obtained if DNA polymerase is preloaded onto the template/primer prior to exposure to nucleotide mixes. The polymerase appears to remain attached to the template/primer even after extensive washing (for example 18 washes with 200 μL buffer before incorporation of the last G).

### Example 6

Purification of Penomic DNA from Whole Blood in a Tip

Control purification: DNA was purified from whole blood (EDTA as anticoagulant) using reagents in the GFX Genomic Blood DNA Purification Kit, Amersham Biosciences. The kit was used to purify DNA from 100 μL fresh, whole blood (EDTA tube) according to the manufacturers instructions, briefly as follows. Extraction solution (500 μL, containing guanidinium isothiocyanate) and 100 μL blood were mixed and incubated at room temperature for 5 minutes. The mix was transferred to a spin column containing a glass-fibre filter and centrifuged in an angle rotor at 5000 g for 1 minute. The filter was washed with 500 μL Extraction Solution and 500 μL wash solution (containing ethanol) by centrifugation. The purified DNA was then eluted by adding 100 μL water heated to 70° C., incubating for 1 minute and centrifuging.

Purification in a tip: A fragment of the glass-fibre filter was packed into a standard 20 μL pipette tip to give a bed volume of approximately 1 μL. Whole blood (2 μL) was mixed with extraction buffer (10 μL) and incubated at room temperature for 5 minutes. The mix was then aspirated into the filter in the pipette tip and ejected. The filter was then washed by aspirating and ejecting 20 μL extraction buffer, and twice with 20 μL washing buffer. DNA was eluted by repeatedly aspirating and ejecting 10 μL Milli-Q water heated to 70° C. The DNA content of the extracts was estimated to 2ng/μL.

Detection of DNA: The DNA purified from the blood was then subject to PCR and Pyrosequencing to check the functioning of the purification methods. The PCR primers were designed to amplify a region around the SNP rs6276 in the monoamine oxidase gene.

Primer sequences (5′-3′):

(SEQ ID NO:5)

| | | |
|---|---|---|
| PCR | B021FPB | Biotin-tgtcaacagcgccgtgaa |

(SEQ ID NO:6)

| | | |
|---|---|---|
| PCR | B022RP | atggagccaagcgaacactg |

(SEQ ID NO:7)

| | | |
|---|---|---|
| Sequencing | B023RS | aag ggt gag gct ggc |

The PCR mix contained 3 pmoles each of the primers B021FPB and B022RP, 200 μM of DATP, dCTP, dGTP and dTTP, 1.5 mM MgCl₂, 3 ng DNA (purified by the GFX kit, the tip or a standard DNA sample prepared from human lymphocytes, Coriell Cell Repositeries at Coriell Institute for Medical Research, USA), and 0.5 U AmpliTaq Gold (Applied Biosystems) in 1×PCR Buffer II (supplied with the enzyme) in a total volume of 15 μL. The mix was subject to thermocycling with the program: 5min, 95° C.; (15s, 95° C.; 30s, 58° C.; 15s, 72° C.)×45; 5 min, 72° C.

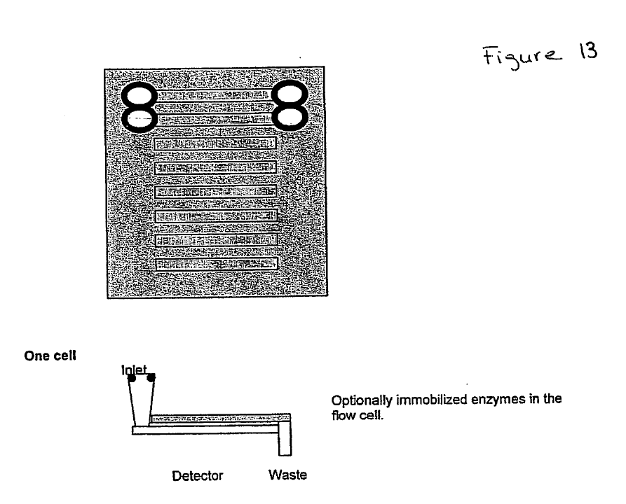

The PCR product was prepared for sequencing on the Pyrosequencing Instrument PSQ™HS 96 according to manufacturers instructions. The PCR products, in 5 μL of reaction mix, were immobilised on Streptavidin Sepharose (Amersham Biosciences), denatured using the Vacuum Prep Tool (Pyrosequencing AB) and the sequencing primer B023RS was annealed. The PCR products were then sequenced (see FIG. 11). The control DNA (FIG. 11 upper panel) had a homozygous C/C genotype. The results for the DNA purified from whole blood using the GFX Genomic Blood DNA Purification Kit (FIG. 11 middle panel) and using the scaled-down method in a tip (FIG. 11 lower panel), were essentially identical and gave a heterozygous (T/C) genotype, with a pyrogram quality comparable to the control DNA. These results indicate that the two purification methods yielded DNA of comparable quality.

### Example 7

Flow Cell for the Reaction Solution, in a Split Reaction

Description of the Flow Cell
The dispensed solution consists of PPi (if primer extension has occurred) in the same buffer used in the previous polymerisation step.
Typical volumes for the added reaction and the enzyme solutions are 0.5 to 5 μl.
The channels of the flow cell have a diameter of about 0.5-1 mm.
The flow in the channel is driven by the flow from the pipette.
The flow is easily replaceable

Sample Cell

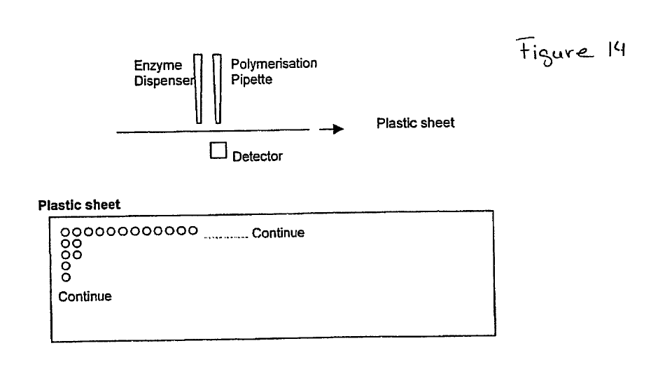

The sample cell consists of a plastic washer with a silvery background for doubling the signals to the detector. The washer is mounted onto a plastic part with grooves, that generates a channel when it is mounted. One channel is used for each sample (see FIG. 13). The number of channels depend on how many samples that are processed at the same time. Each channel has an inlet well and a waste (see FIG. 13). Each well has a sealing on the inlet for the pipette, so that it is possible to use the pipette to push solutions through the channels. The channels may also be capillary tubings. One advantage with a set-up like this is that the volume of the flow cell is defined.

Method

1. Add the reaction solution from the polymerisation step to the inlet of the cell by using the polymerisation pipette.
2. Add the necessary enzymes for the detection, i.e. luciferase, luciferin, APS and sulphurylase in a buffer by using the polymerisation pipette.
3. Let the reagents of step 1 and 2 mix for a few seconds.
4. Use the pipette to press the solution through the channel.
5. Detect the possible light production (if Ppi present) with a suitable detector.
6. Wash the polymerisation pipette and the channel with distilled water, optionally supplemented with apyrase. The detector is now prepared for a new addition according to step 1.

### Example 8

Detector Using a Plastic Film for the Reaction Solution in a Split Reaction

Description:
The dispensed solution consists of PPi (if a primer extension has occurred) in the same buffer used in the previous polymerisation step.
Typical volumes for the added reaction and the enzyme solutions are 0.5 to 5 μl.
The spots has a diameter of about 1-5 mm

The detection unit

The detection unit consists of a detector and either (1) a plastic sheet that is coated with freeze-dried enzyme as spots in rows (depending on the number of samples that are processed at the same time), or (2) with a dispenser that dispenses the enzyme spots to generate the same kind of pattern described in (1).

Detector arrangement with enzyme dispenser—see FIG. 14.

Plastic sheet—see FIG. 14. The plastic sheet may also be a microtiter plate or be equipped with small wells. Preferably, the plastic sheet is movable by a motor.

Method (Using the Dispensing Alternative (2))
1. Dispense the enzyme on a spot on the plastic sheet.
2. Move the enzyme spot under the polymerisation pipette.
3. Add the reaction solution from the polymerisation step to the enzyme spot, using the polymerisation pipette,
4. Detect the possible light production (if Ppi present) with a suitable detector.

### Example 9

Pre-PCR and Post-PCR Modules

In one example, pre-PCR and post-PCR-modules are integrated in one system, which preferably is automated. All steps is possible to perform using a pipetting robot. The entire chain of steps is according to an example the following:

Pre-PCR:

(1) Use a pipette tip, such as a standard tip, to mix blood sample with extraction buffer in a well.

(2) Use a tip with means for binding nucleic acid molecules for aspirating the extract of step (1) from the well to the tip and bind DNA. Wash the tip.

(3) Aspirate elution buffer into the tip of step (2) and elute DNA into a well in a PCR plate.

(4) Use a standard tip to add amplification reagents to the well in the PCR-plate.

Post-PCR:

(5) Subject the PCR-plate to thermocycling in order to amplify the DNA.

(6) Use a standard tip to mix PCR-product with binding buffer. Immobilise the PCR-product by aspirating the PCR-product from the well to a tip comprising a streptavidin solid-phase.

(7) Denature bound DNA and wash the bound single-stranded DNA using reagents in a tray.

(8) Add annealing buffer and primer for a subsequent primer extension reaction. Heat the tip to 60° C. and cool it, in order for the primer to anneal to the bound single-stranded DNA template.

(9) Eject excess primer from the tip and wash the primer template complex.

(10) Aspirate pyrosequencing reagents and detect any generated light in situ, by using a detector.

(11) Wash the tip.

(12) Repeat steps (10) and (11) in order to sequence the template.

References

Agrawal, S., Christodoulou, C. and Gait, M. J. (1986) Efficient methods for attaching non-radioactive labels to the 5′ ends of synthetic oligodeoxyribonucleotides. Nucleic Acids Res. 14 6227-6245.

Alderborn, A., Kristofferson, A., Hammerling, U. (2000) Determination of single nucleotide polymorphisms by real-time pyrophosphate DNA sequencing Genome Res. 10:1249-1258.

Borcherding H, Hicke H G, Jorcke D, Ulbricht M (2003) Affinity membranes as a tool for life science applications. Ann N Y Acad Sci. 984 470-9.

Chapman-Smith,A and Cronan, J E (1999) Molecular biology of biotin attachment to proteins. J. Nutr. 129 477S-484S.

Eckert, K A and Kunkel T A (1993). Effect of reaction pH on the fidelity and processivity of exonuclease-deficient Klenow polymerase., J. Biol. Chem. 268 13462-13471.

Ehn, M (2003) “Protein based approaches for further development of the pyrosequencing technology platform”, Doctoral thesis, Dept. of Biotechnology, Royal Institute of Technology, Stockholm.

Langer, P. R., Waldrop, A. A., and Ward D. C. (1981) Enzymatic synthesis of biotin-labeled polynucleotides: novel nucleic acid affinity probes. Proc. Natl. Acad. Sci. 78 6633-6637.

Nyren, P. (2001) Method for sequencing DNA based on the detection of the release of pyrophosphate and enzymatic nucleotide degradation; Patent: US-B1-6258568, WO 98/28440 Ronaghi, M., Uhlen, M., and Nyren, P. (1998) A sequencing method based on real-time pyrophosphate. Science 281, 363-365.

Sambrook, J. and Russell, D W (2001) Molecular Cloning Laboratory Manual, 3^{rd} edition, Cold Spring Harbor Laboratory Press 2001.

Zhao, Z. and Ackroyd, J. (1999) A biotin phosphoramidite reagent for the automated synthesis of 5′-biotinylated oligonucleotides. Nucleosides Nucleotides 18 1231-1234.

1. A tubular member having therethrough an axial bore one end of which is releasably insertable over the nozzle of a pipettor to connect said member to said pipettor, wherein the bore comprises means for binding nucleic acid molecules for a subsequent luminescent biomolecular reaction, wherein the means for binding nucleic acid molecules comprises at least one filter or membrane positioned in the bore, on which filter or membrane solid-phase beads for binding the nucleic acid molecules are positioned.

2. The tubular member of claim 1 wherein the tubular member is a pipette tip.

3. The tubular member of claim 1 whereby the tubular member further is adapted to allow the detection of a luminescent biomolecular reaction occurring in the tubular member.

4. The tubular member of claim 1 wherein the solid-phase beads are streptavidin-coated Sepharose beads.

5. The tubular member of claim 1 whereby the tubular member has a transparency of at least 10%, at least 50%, at least 80% or at least 90%.

6. The tubular member of claim 1 wherein at least one enzyme is immobilized to the solid phase beads.

7. The tubular member of claim 6 wherein the enzyme is sulphurylase or luciferase.

8. A method for using the tubular member of claim 1 comprising the step of immobilising a nucleic acid sample in the tubular member and performing an enzymatically based reaction involving the nucleic acid sample.

9. The method of claim 8 wherein the enzymatically based reaction comprises a polymerase reaction step.

10. A method for performing a sequencing by synthesis reaction in a tubular member comprising the steps of:
(a) immobilising a template nucleic acid sample to the tubular member of claim 1 and annealing an oligonucleotide primer to the template nucleic acid wherein said oligonucleotide primer hybridizes to a predetermined position on the template;
(b) extending the oligonucleotide primer with a polymerase by one nucleotide directly adjacent to its 3′-end in a reaction mixture, whereby the nucleotide that extends the primer is complementary to the template nucleic acid in this position; and
(c) detecting the identity of the nucleotide that extends the primer.

11. The method of claim 10 further comprising repeating said steps (b) and (c) for a desired number of cycles.

12. The method of claim 10 wherein an enzyme mix, comprising nucleic acid polymerase, sulphurylase and luciferase, substrate, comprising APS and luciferin, and a dNTP is aspirated into the tubular member under conditions allowing extension of the oligonucleotide primer if the chosen dNTP is complementary to the template in the position directly adjacent to the 3′-end of the primer.

13. Method according to claim 10, wherein said detecting step comprises monitoring for a presence of a light signal by a light detector positioned above the tubular member or on a side of the tubular member.

14. Method according to claim 10, wherein said detecting step comprises monitoring for a presence of a light signal by a light detector inside the tubular member.

15. Method according to claim 10, wherein said detecting step comprises monitoring for a presence of a light signal generated within said tubular member by a light detector positioned below the tubular member.

16. Method according to claim 10, wherein said detecting step comprises monitoring for a presence of a light signal generated within said tubular member by detecting a light signal after ejecting at least a part of the reaction mixture.

17. A method for performing a sequencing by synthesis reaction in a tubular member comprising means for binding nucleic acid molecules of claim 1 comprising the steps of:
(a) immobilising a template nucleic acid sample to the tubular member of claim 1 and annealing an oligonucleotide primer to the template nucleic acid template wherein said oligonucleotide primer hybridizes to a predetermined position on the template to form an immobilized template/primer complex;
(b) extending the oligonucleotide primer with a reaction mixture which contains a nucleic acid polymerase and one nucleotide directly adjacent to its 3′-end, whereby the nucleotide that extends the primer is complementary to the template nucleic acid in this position;
(c) dispensing at least part of the reaction mixture of step (b) into a means for detection;
(d) detecting the identity of the nucleotide that extends the primer in the means for detection.

18. The method of claim 17 further comprising repeating said steps (b) to (d) for a desired number of cycles.

19. The method of claim 17 wherein said reaction mixture comprises a nucleic acid polymerase and a dNTP and wherein said reaction mixture is aspirated into the pipette tip under conditions allowing extension of the oligonucleotide primer if the dNTP is complementary to the template in the position directly adjacent to the 3′-end of the primer.

20. Method according to claim 17 wherein step (b) is performed at a temperature between about 37° C. to 72° C., and step (d) is performed at a temperature between 25° C. to 28° C.

21. Method according to claim 17 wherein the means for detection is a detection solution comprising sulphurylase, luciferase, APS and luciferin and wherein in step (b) said detection solution and said reaction mixture are mixed thereby allowing a light signal to develop in case of a primer extension, wherein said light signal is detected using a light detection device.

22. The method of claim 21 wherein said light detection device is a CCD camera, phototransistor or photomultiplier tube.

23. Method according to claim 17 wherein said means for detection comprises a flow cell comprising a detection solution comprising sulphurylase, luciferase, APS and luciferin, whereby the dispensed reaction mixture of step (b) and the detection solution are mixed, thereby allowing a light signal to develop in case of a primer extension, which light signal is detected from within the flow cell using a light detection device.

24. Method according to claim 17, wherein the means for detection is a film or a well comprising a freeze-dried detection reagent selected from the group consisting of sulphurylase, luciferase, APS, luciferin and a combination thereof, and whereby the freeze-dried detection reagents are mixed with the sequencing by synthesis reaction in step (b) and thereby allowing a light signal to develop if there is a primer extension, which light signal is detected from the film or well using a light detection device.

25. Method according to claim 17, wherein the means for detection is a film or a well, comprising a detection solution comprising sulphurylase, luciferase, APS and luciferin, whereby the dispensed reaction mixture of step (b) and the detection solution are mixed, thereby allowing a light signal to develop in case of a primer extension, which light signal is detected from the film or well using a phototransistor, CCD camera, photomultiplier tube or other light detection device.

26. Method according to claim 17, wherein the means for detection comprises a capillary detection chamber comprising freeze-dried detection reagents comprising sulphurylase, luciferase, APS and luciferin, and whereby a part of the reaction mixture of step (b) is allowed into the capillary detection chamber, whereby the dispensed reaction mixture of step (b) and the freeze-dried detection reagents are mixed, thereby allowing a light signal to develop in case of a primer extension, which light signal is detected from the capillary detection chamber using a phototransistor, CCD camera, photomultiplier tube or other light detection device.

27. Method according to claim 17, in which the sulphurylase, luciferase, or both the sulfurylase and luciferase are immobilised in the means for detection, and whereby the dispensed reaction mixture of step (b) is mixed with APS, luciferin and optionally said immobilized sulphurylase, immobilized luciferase, or immobilized sulfurylase and immobilized luciferase, and is flowed through the immobilised enzyme(s), thereby allowing a light signal to develop in case of a primer extension, which light signal is detected from a light detection device.

28. Method according to claims 17, wherein the immobilised template/primer complex is exposed to polymerase before primer extension is initiated, wherein the polymerase binds to the template/primer complex and remains bound for the remaining cycles of the sequencing reaction.

29. Method according to claim 18, wherein the tubular member is washed with a washing buffer comprising apyrase between each reaction cycle.

30. Method according to claim 17, wherein apyrase is present in the means for detection.

31. Method according to claim 17, wherein the template nucleic acid sample is prepared by means of purifying the template nucleic acid sample by the use of a tubular member that comprises means for binding nucleic acid molecules and amplifying the purified sample before it is immobilised in the tubular member for performing a sequencing by synthesis reaction.

32. The method of claim 31 wherein said tubular member is a pipette tip.

33. The method of claim 31 wherein said means for detection comprises immobilized luciferase, sulfurylase, or a combination thereof.

34. An automated system for performing the method according to claim 20, comprising at least one pipettor allowing at least one said tubular member to be releasably insertable over the nozzle of the pipettor to connect said tubular member to said pipettor, and means for controlling the system.

35. A kit for performing a sequencing reaction comprising:
(a) the tubular member of claim 1; and
(b) reagents for performing primer extension or reagents for performing pyrophosphate detection.

36. A kit for performing a sequencing reaction comprising:
(a) the tubular member of claim 1 for performing a sequencing-by-synthesis reaction;
(b) a pipette tip adapted for purifying the template nucleic acid sample to be used in a subsequent sequencing-by-synthesis reaction; and
(c) reagents for performing primer extension or reagents for performing pyrophosphate detection.

37. Computer program for causing a computerised apparatus to determine the identity of at least one nucleotide in a nucleic acid molecule, by way of sequentially detetcing a measurable quantity of a reaction when the nucleotide is incorporated to form a base pair with the complementary base in the nucleic acid molecule, comprising a computer readable code means which when run causes the computerised apparatus to perform the method steps according to claims 1.

38. Computer program product for causing a computerised apparatus to determine the identity of at least one nucleotide in a nucleic acid molecule, by way of sequentially detecting a measurable quantitiy of a reaction when the nucleotide is incorporated to form a base pair with the complementary base in the nucleic acid molecule, comprising a computer readable medium, and a computer program according to claim 37, the computer program being recorded on said computer readable medium.


SEQUENCE LISTING (7 sequences)

SEQ ID NO: 1 - DNA, 9 bp, Artificial
  Synthesized Oligonucleotide
ytgctctga

SEQ ID NO: 2 - DNA, 32 bp, Artificial
  Synthesized Oligonucleotide
gtaaaacgacggccagtctgacgaattccagc

SEQ ID NO: 3 - DNA, 35 bp, Artificial
  Synthesized Oligonucleotide
caacattttgctgccggtcagactgcttaaggtcg

SEQ ID NO: 4 - DNA, 120 bp, Artificial
  Synthesized Oligonucleotide
tcagcagacccatagccttctccctgccttcctctgccctccctccagcaggtggcacagacccttccctgtctcccttggcggatggtcccygctctgagtgcagcggatagggtccca

SEQ ID NO: 5 - DNA, 22 bp, Artificial
  Synthesized Oligonucleotide
tcagcagacccatagccttctc

SEQ ID NO: 6 - DNA, 13 bp, Artificial
  Synthesized Oligonucleotide
ggcggatggtccc

SEQ ID NO: 7 - DNA, 19 bp, Artificial
  Synthesized Oligonucleotide
tgggataggcgacgtgagt